(19) United States
(12) Patent Application Publication
(10) Pub. No.: US 2019/0030208 A1
(43) Pub. Date: Jan. 31, 2019

(54) SURFACE MODIFIED STRUCTURE FOR IMPROVING HEMOCOMPATIBILITY OF BIOMEDICAL METALLIC SUBSTRATES

(71) Applicant: NATIONAL CHUNG HSING UNIVERSITY, Taichung City (TW)

(72) Inventors: Shu-Ping Lin, Taichung City (TW); Pei-Chieh Wong, Taichung City (TW)

(21) Appl. No.: 16/152,399

(22) Filed: Oct. 4, 2018

Related U.S. Application Data
(63) Continuation-in-part of application No. 15/256,751, filed on Sep. 6, 2016, now abandoned.

(30) Foreign Application Priority Data
Sep. 9, 2015 (TW) 104129830

(51) Int. Cl. A61L 27/34; A61L 31/10; A61F 2/82; A61L 31/02

(52) U.S. Cl. CPC A61L 27/34; A61L 31/10; A61L 2400/12; A61L 31/022; A61L 2400/18; A61F 2/82

(57) ABSTRACT

The present invention relates to a surface modification method for improving the hemocompatibility of a biomedical metallic substrate, comprising: fixing a sulfur-containing and nitrogen-containing monolayer film on the surface of oxide layer of the biomedical metallic substrate by molecular self-assembly. The surface modification improves the hydrophilicity and hemocompatibility of the biomedical metallic substrate in contact with the blood, and ensures that the biomedical metallic substrate is non-toxic to the endothelial cells.

### CROSS-REFERENCE TO RELATED APPLICATIONS

The present application is a continuation-in-part of U.S. application Ser. No. 15/256,751 filed Sep. 6, 2016. The U.S. application Ser. No. 15/256,751 claims benefit of priority of the Taiwan Patent Application No. 104129830, filed Sep. 9, 2015. The entirety of said applications is incorporated by reference herein.

### BACKGROUND OF THE INVENTION

### Technical Field of the Invention

The present invention relates to a surface modification technique for improving the biocompatibility, especially the hemocompatibility, of biomedical metallic substrates. Particularly, the present invention relates to a modification with sulfur-containing and nitrogen-containing monolayer film on the surface of metallic materials.

### Background

The known biomedical metallic materials include platinum, gold, tungsten, rhenium, palladium, rhodium, ruthenium, titanium, nickel, iridium and alloys of these metals, such as stainless steel, titanium/nickel alloy, and platinum iridium alloy. Such metallic materials can be made into the implant for contact with living tissue or long-term exposure to the blood, and can also be used as a surface coating material to other substrates. The implant for exposing to the blood should possess superior hemocompatibility.

In the case of vascular stents, the bare metallic stent is typically made of 316L stainless steel, cobalt-based alloys, titanium or tantalum. The drug-eluting stents are coated with drug-containing coating on the surface of the metal stent, for sustained release of the drugs in the coating to the bloodstream. The coating may be a polymeric coating, such as polyethylene-co-vinyl acetate (PEVA), poly n-butyl methacrylate (PBMA), and the like. The drug contained in the coating may be an anticoagulant, such as heparin, or a drug inhibiting smooth muscle cell growth, such as sirolimus and paclitaxel (Taxol).

WO2014169281 discloses a vascular stent coated with polyelectrolyte multilayers of a polycation and a polyanion. The polycation may be chitosan. The polyanion may be a glycosaminoglycan. At least one of the polycation or polyanion may include nitric oxide-releasing groups. The medical device may release nitric oxide from the surface for the purpose of reducing platelet activation. The medical device may further include a growth factor adsorbed on at least one of the polyelectrolyte layer. The growth factor may be vascular endothelial growth factor (VEGF).

US20130224795 discloses a method for fixing a bioactive molecule onto a substrate surface by using polyphenol oxidase. In the presence of polyphenol oxidase, a bioactive molecule containing a phenol or catechol group can be simply in situ oxidized within a short time to dopa or dopaquinone which forms a coordinate bond with a metal or polymer substrate, thus stably immobilizing the bioactive molecule onto the substrate surface. The bioactive molecules include cell adhesion peptides, growth factors, growth hormones, proteins, anti-thrombotic agents, and endothelialization inducing agents. The cell adhesion peptides and growth factors can be simply immobilized to medical metal or polymer substrate surfaces such as orthopedic or dental implants. Also, antithrombotic agents and/or endothelialization inducing agents may be immobilized to medical substrates for vascular systems, such as stents and artificial blood vessels.

Coating technology has been widely used in the surface modification of biomedical substrates. However, the coated film is physically attached to the surface of a biomedical substrate, and the stability of physical binding is relatively weaker than the immobilization of bioactive molecules. As for the immobilization technique of bioactive molecules, its disadvantages are the technical complexity and taking a long period of time to achieve the chemical reactions for the immobilization. Additionally, it is difficult to remove or control the production and side effects of the byproducts from the bioactive molecules.

### SUMMARY OF INVENTION

The purpose of the invention is to provide a surface modification method for improving the hemocompatibility of biomedical metallic substrate, comprising forming a sulfur-containing and nitrogen-containing monolayer film on the surface of oxide layer of the biomedical metallic substrate by molecular self-assembly. The surface modification will improve the hydrophilicity and hemocompatibility of the biomedical metallic substrate in contact with the blood and ensure that the biomedical metallic substrate is non-toxic to the endothelial cells.

The surface modification method for improving the hemocompatibility of biomedical metallic substrate first comprises: immobilizing a sulfur-containing monolayer film on the surface of oxide layer of the biomedical metallic substrate by molecular self-assembly.

In certain embodiments of the invention, the biomedical metal is preferably a titanium or titanium alloy. The oxide layer may be a native oxide or an oxide layer created by a surface modification technique. The molecular self-assembly comprises: contacting the biomedical metallic substrate having an oxide layer with a solution of a silanol chemical derivative containing mercapto group or sulfur atom for a predetermined period of time, and immobilizing a sulfur-containing monomolecular film to expose functional mercapto group or sulfur atom on the surface of the oxide layer by self-assembly.

The modification of the sulfur-containing and nitrogen-containing monolayer film on the surface of oxide layer of the biomedical metallic substrate using oxygen and nitrogen will confer hydrophilic and hemocompatible properties to the substrate. As used herein, “hemocompatibility” refers to the blood clotting time after contacting with the biomedical metallic substrate, including prothrombin time (PT) and activated partial thromboplastin time (aPTT), being in a normal range, and lowered fibrinogen concentration of the contacting substrate surface. In addition, there is no platelet activation or erythrocyte adhesion occurring in the blood contacting surface, and the substrate is non-toxic to the endothelial cells.

“PT and aPTT are in a normal range” means the biomedical metallic substrate of the present invention does not adversely affect the exogenous coagulation pathway and endogenous coagulation pathway of blood, but can maintain the dynamic equilibrium between blood coagulation and anti-coagulation.

Fibrinogen is a glycoprotein in vertebrates that helps in the formation of blood clots. Thromboplastin, released from damaged platelets, converts prothrombin to thrombin in the action of calcium ion. Thrombin coagulates the originally water-soluble fibrinogen into water-insoluble fibrin. Fibrin links other blood cells into aggregation and becomes solidified blood clot. The substrate of the present invention would reduce the fibrinogen concentration in the contacted blood, so that the fibrin cannot be produced to entangle blood cells, and further ensure that no blood clots are formed on the substrate surface. Therefore, the expected physiological effect of lowering fibrinogen concentration is to prevent the formation of blood clot.

The platelet activation will promote greater blood coagulation. According to the present invention, platelet activation will not occur in the blood that contacts with the substrate surface, which ensures that no blood clots are formed on the substrate surface.

Erythrocyte adhesion easily leads to the abnormal aggregation of blood cells, which becomes the base for thrombosis. No erythrocyte adhesion occurs on the substrate surface of present invention. The expected physiological effect is not inducing thrombus formation.

Vascular endothelial cells ensure the integrity of vascular wall and can promote natural healing of the vascular wall. The incomplete or delayed healing of vascular endothelium will result in the highly exposed extracellular matrix, which activates the coagulation and leads to thrombosis. The substrate of present invention is totally non-toxic to the endothelial cells and allows endothelial cells to grow normally on the substrate surface. The expected physiological effects neither damage the endothelial cells nor activate the coagulation and thrombosis.

The surface modification method for improving the hemocompatibility of biomedical metallic substrate further comprises forming a layer of sulfur-containing and nitrogen-containing functional groups of monolayer film that is fixed on the surface of the biomedical metallic substrate. The sulfur-containing functional groups include sulfite (—SO₃H), mercapto (—SH) and S-Nitrosothiols (—SNO). A disulfide bonds (—S—S—) can be formed by two close mercapto groups. Since the functional groups of —SH on MPTMS are close enough, the two —SH functional groups would form a disulfide bond. The nitrogen-containing groups include nitrate (—NO₃H) and nitric oxide (—NO).

By the creation of highly negative charged surface of sulfur-containing and nitrogen-containing groups of monolayer film, the occurrence of platelet activation and blood cell adhesion on the substrate of present invention will be reduced.

The present invention provides the formation of highly negative charged surface for improving the hemocompatibility of biomedical metallic substrate. Relative to the active molecule fixing technology, the method of the present invention requires a short reaction time, is easy to operate, and does not produce any by-product that is difficult to remove or control.

### BRIEF DESCRIPTION OF THE DRAWINGS

FIG. 1 illustrates the chemical structure of an anodized titania nanotube (ATN) substrate surface modified with a monolayer film consisting of sulfur-containing silanol functional groups.

FIG. 2 shows the results of hydrophilicity evaluation of the four samples described in Example 1 by droplet angle goniometry.

FIG. 3 shows the field-emission scanning electron microscope (FESEM) graphs of the platelet-adhered surface of the four samples described in Example 1.

FIG. 4 shows the FESEM graphs of the erythrocyte-adhered surface of the four samples described in Example 1.

FIG. 5 shows the fluorescent staining of endothelial cells cultured on the surface of the four samples described in Example 1.

FIG. 6 shows the quantitative analyses of endothelial cell numbers cultured on the surface of the four samples described in Example 1.

FIG. 7 shows the electron spectroscopy for chemical analysis (ESCA) N₁ₛ spectra of the modified substrates of Examples 2 and 3.

FIG. 8 shows the ESCA S_{2p3/2} spectra of the modified substrates of Examples 2 and 3.

FIG. 9 shows the Fourier-transform infrared spectroscopy (FTIR) spectra of the modified substrates of Examples 2 and 3.

FIG. 10 shows the depth profile of main atomic concentrations of the surface analyses of the modified substrates of Examples 2 and 3 in ESCA.

### DETAILED DESCRIPTION OF THE INVENTION

The characteristics and advantages of the present invention will be further illustrated and described in the following examples. The examples described herein are intended to illustrate the invention, and not to limit the scope of the invention.

The surface modification of the present invention for improving the hemocompatibility of titanium- or titanium alloy-based biomedical metallic substrate comprises: forming a sulfur-containing and nitrogen-containing monolayer film on the surface of oxide layer of a biomedical metallic substrate by molecular self-assembly.

The oxide layer of the biomedical metallic substrate may be a native oxide or an oxide layer created by a surface modification technique. In the present invention, the oxide layer is created on the surface of a titanium or titanium alloy substrate by an anode oxidation process or gas plasma surface treatment. The oxide layer provides the chemical bonding required in the subsequent molecular self-assembly. In the present invention, the titanium substrate with the oxide layer is abbreviated as the ATN (anodized titania nanotube) substrate, which is described in some journals, for example, “Investigation of the Interfacial Effects of Small Chemical-Modified TiO₂ Nanotubes on 3T3 Fibroblast Responses. Shu-Ping Lin, et al., ACS Appl. Mater. Interfaces 2014, 6, 12071-12082.”

In Example 1 of the present invention, the steps of said molecular self-assembly comprise: immersing the ATN substrate in a 0.1%-20% solution of a silanol chemical derivative containing mercapto group for a period of 10 minutes to 24 hours. During the immersion, the molecular self-assembly is performed on said oxide layer through the silanol group, and the sulfur-containing functional groups are then exposed on the outermost surface of the metals.

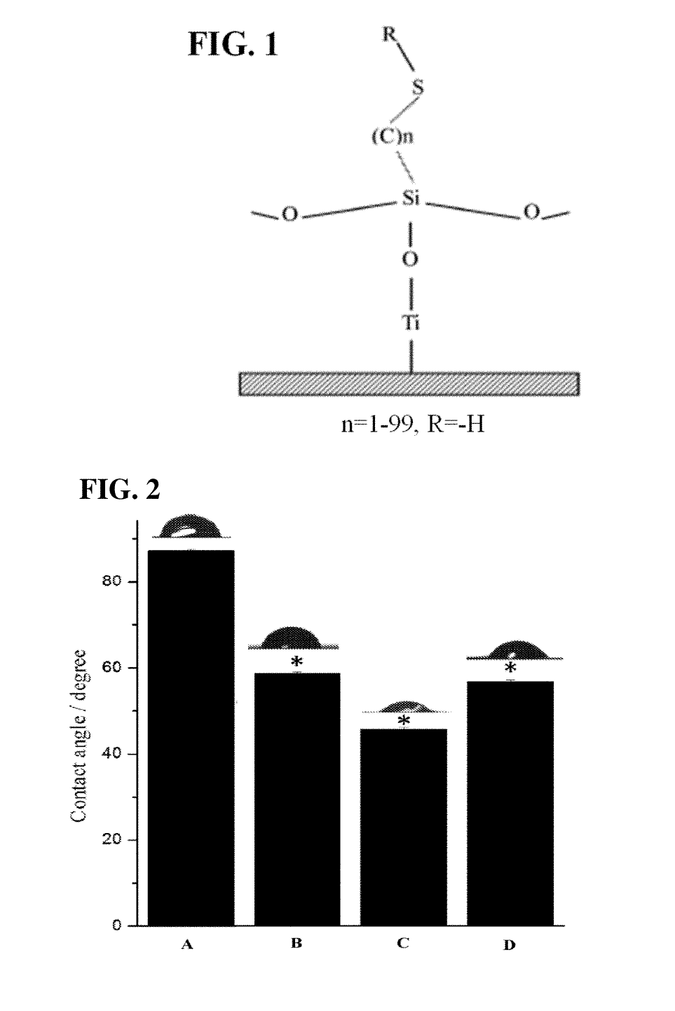

As shown in FIG. 1, the sulfur-containing functional group (—SH) was immobilized on the surface of the ATN substrate by the silanol group binding with titanium (—Ti—O—Si—). Additionally, a further nitric oxide (NO) modification is formed on the surface of the sulfur-containing and nitrogen-containing monolayer film by plasma treatment. Alternately, the nitric oxide (NO) modification is formed on the surface of the ATN substrate, and a sulfur-containing monomolecular film is formed on the surface of the nitric oxide (NO) modification by molecular self-assembly.

Four samples include: A, titanium substrate (Ti); B, ATN substrate modified with 3-mercaptopropyltrimethoxysilane (MPTMS) (MPTMS-ATN); C, outermost NO-coating on MPTMS-modified ATN substrate (NO-MPTMS-ATN); and D, outermost MPTMS modification on NO-coated ATN substrate (MPTMS-NO-ATN). The substrate A is a control, and substrates B, C and D are exemplary substrates of present invention.

The hydrophilicity of the four samples was evaluated by droplet angle goniometry. Results are shown in FIG. 2. The contact angle of substrate A (Ti) was almost 90°, indicating it was hydrophobic. The contact angles of substrates B (MPTMS-ATN), C (NO-MPTMS-ATN) and D (MPTMS-NO-ATN) were less than 60°, indicating they were relatively more hydrophilic.

### Blood Testing

The four substrates were incubated with fresh blood respectively to investigate the coagulation and anticoagulation actions of the substrates. Platelet-poor plasma (PPP), platelet-rich plasma (PRP) and red blood cells (RBCs) were isolated from fresh blood by centrifugation. The PPP was contacted with the four substrates and incubated at 37° C. in CO₂ incubator for one hour, then subjected to the tests of prothrombin time (PT) and activated partial thromboplastin time (aPTT), and the measurement of fibrinogen concentration were taken. Furthermore, the four substrates were contacted with PRP and RBC and incubated at 37° C. in CO₂ incubator for one hour, then the adhesion of platelet or blood cell on the surface of four substrates were observed by field-emission scanning electron microscope (FESEM).

The results of PT, aPTT and fibrinogen concentration analyses are listed in Table 1. It is shown that the PT and aPTT values of the four substrates are in the normal range, indicating that no negative effects on the exogenous coagulation pathway and endogenous coagulation pathway of blood were produced, and the dynamic equilibrium between blood coagulation and anti-coagulation was maintained. The fibrinogen concentrations on the surface of the four substrate groups were all lower than the normal value, and no blood clotting was observed on the surface of the substrates.

TABLE 1

| | | | fibrinogen |
|---|---|---|---|
| | | | concentration |
| | PT (sec) | aPTT (sec) | (mg/dL) |
| Normal range | 8.0~12.0 | 23.9~35.5 | 200.0~400.0 |
| Substrate A (Ti) | 11.08 ± 0.08 | 29.36 ± 0.51 | 195.64 ± 3.28 |
| Substrate B | 11.27 ± 0.4 | 29.23 ± 0.59 | 191.27 ± 5.93 |
| (MPTMS-ATN) | | | |
| Substrate C | 11.3 ± 0.2 | 29.8 ± 0.7 | 189.23 ± 5.03 |
| (NO-MPTMS-ATN) | | | |
| Substrate D | 11.17 ± 0.31 | 29.33 ± 0.31 | 188.67 ± 2.04 |
| (MPTMS-NO-ATN) | | | |

The observed results of platelet adhesion on the surface of the four substrates by FESEM are shown in FIG. 3. Platelets were adsorbed on the substrate A (Ti), while no platelets were adsorbed on the substrates B (MPTMS-ATN), C (NO-MPTMS-ATN) and D (MPTMS-NO-ATN). Platelet activation occurred on the substrate A, but no platelet activation occurred on the substrates B, C and D of the present invention. The platelet activation will prime blood coagulation. Platelet activation did not occur on the surface of substrates B, C and D of the present invention therefore no blood clots are formed on the substrate surface.

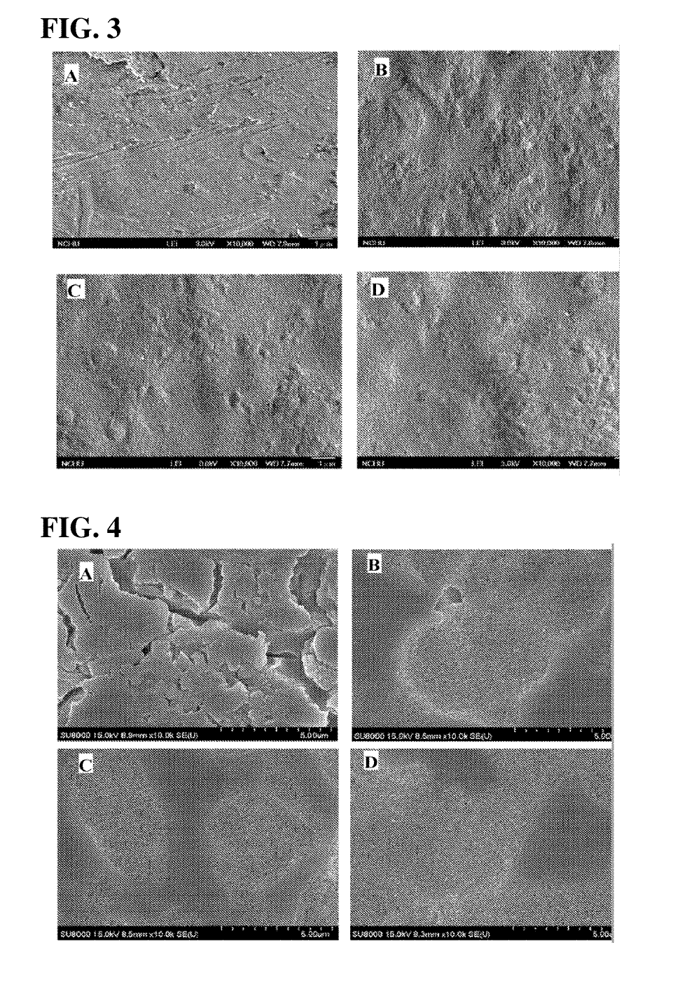

The observed results of red blood cells (RBCs) adhesion on the surface of the four substrates by FESEM are shown in FIG. 4. There was no red blood cell adsorbing on the surface of the four substrates. The expected physiological effect of no RBC adhesion on the surface of the present substrates is that no thrombus formation is induced.

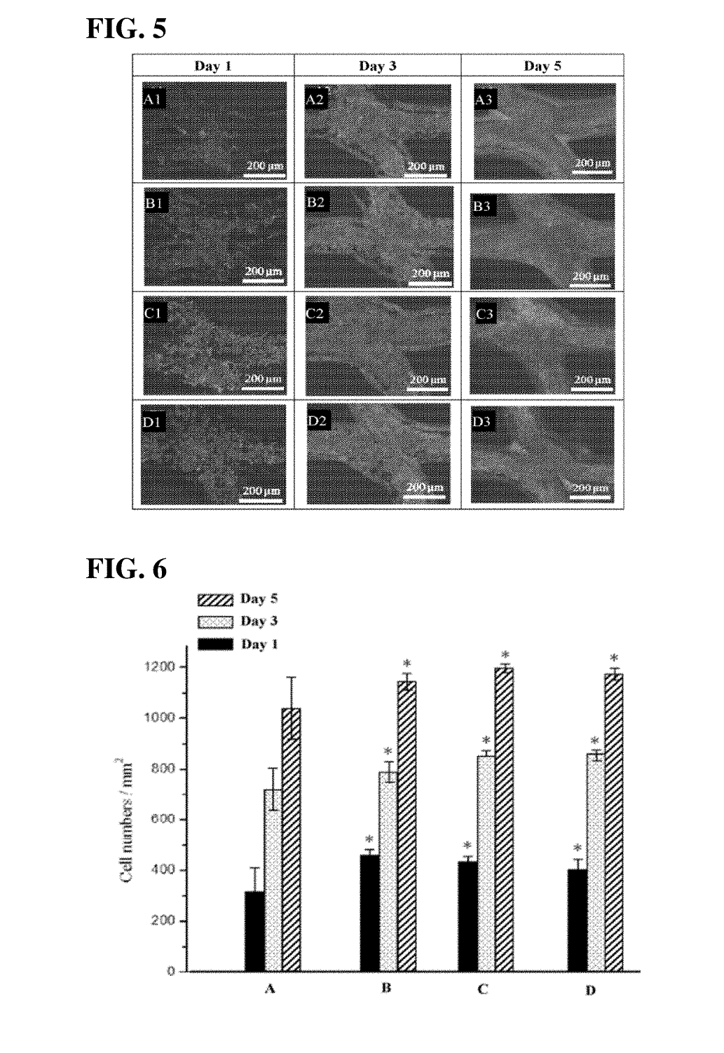

For the vascular endothelium test, vascular endothelial cells were attached to the surface of the four substrates. The nucleus of endothelial cell was stained with the fluorescein dye 4′,6-diamidino-2-phenylindole (DAPI), and the staining result was observed using a fluorescence microscope. The growth of endothelial cell on the surface of the four substrates at Day 1, Day 3 and Day 5 are shown in FIG. 5. FIG. 6 shows the statistic analyses of the number of growing endothelial cells. The number of endothelial cells increased progressively with the days, especially the cell numbers were greater in the substrate B, C and D groups than in the substrate A. The results indicate that the substrates B, C and D of the present invention were non-toxic to endothelial cells, promising the normal growth and proliferation of endothelial cells on the substrate surface. The expected physiological effect is no activation of the coagulation and thrombosis.

On the basis of Example 1, the present invention is further studied by adjusting operation conditions as in Examples 2 and 3.

In Example 2, the ATN substrate is immersed in a MPTMS solution having a volume concentration of 1% for 1 hr to form a MPTMS-ATN substrate. The substrate was washed with absolute alcohol to remove unreacted MPTMS and then dried in an oven. The MPTMS-ATN substrate was then treated with the O₂/N₂ plasma at 2×10⁻² torr for 1 min in a RF plasma reactor. The flow rates were 1-10 sccm for O₂ and 1-10 sccm for N₂ gas. The resultant substrate was used in the following tests as sample E.

In Example 3, the ATN substrate is treated with the O₂/N₂ plasma at 2×10⁻² torr for 1 min in a RF plasma reactor to form a NO-ATN substrate. The flow rates were 1-10 sccm for O₂ and 1-10 sccm for N₂ gas. The NO-ATN substrate was then immersed in a MPTMS solution having a volume concentration of 1% for 1 hr. The modified substrate was then washed with absolute alcohol to remove unreacted MPTMS and then dried in an oven. After that, the resultant substrate was used in the following tests as sample F.

### Electron Spectroscopy for Chemical Analysis (ESCA)

Then the modified substrates (samples E and F) produced in Examples 2 and 3, respectively, were analyzed using ESCA. In the surface survey, the ratio of oxygen to Ti increased after anodization, which indicates that a dense titanium oxide layer is formed. E and F substrates modified with MPTMS and O₂/N₂ plasma showed the sulfur, silicon, and nitrogen peaks in the narrow scan. Compared with the only anodized group, it significantly shows a characteristic peak of amine group in the N₁, region. These mean that the surface treatment and the chemical modification are successful.

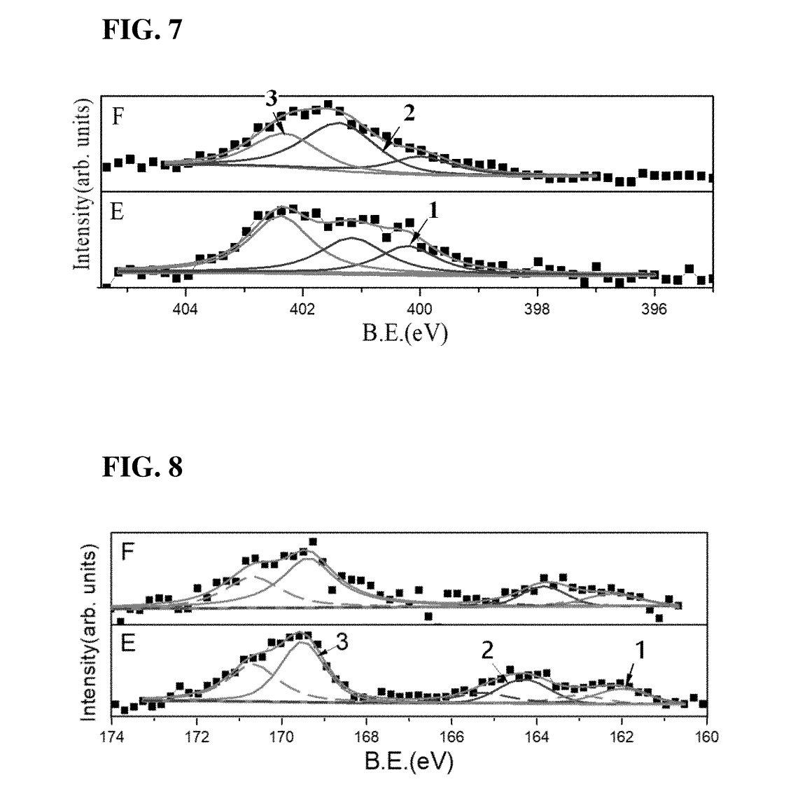

FIG. 7 shows the N₁ₛ scan, the peak 1 for the nitric titanium group (Ti—N) is resolved at 399.9 eV. The peak 2 for the nitric oxide group (—NO) is resolved at 401 eV. The peak 3 for the nitrate (—NO₃⁻) and the group (—O═N) are resolved at 402.4 eV.

FIG. 8 shows the S₂ₚ₃₁₂ scan, the peak 1 for the mercapto group (—SH) is resolved at 162.3 eV. The peak 2 for the disulfide bond (—S—S—) and the S-Nitrosothiols (—SNO) are resolved at 164.2 eV. The peak 3 for the sulfite (—SO₃⁻) is resolved at 169 eV.

### Fourier-Transform Infrared Spectroscopy (FTIR) Analyses

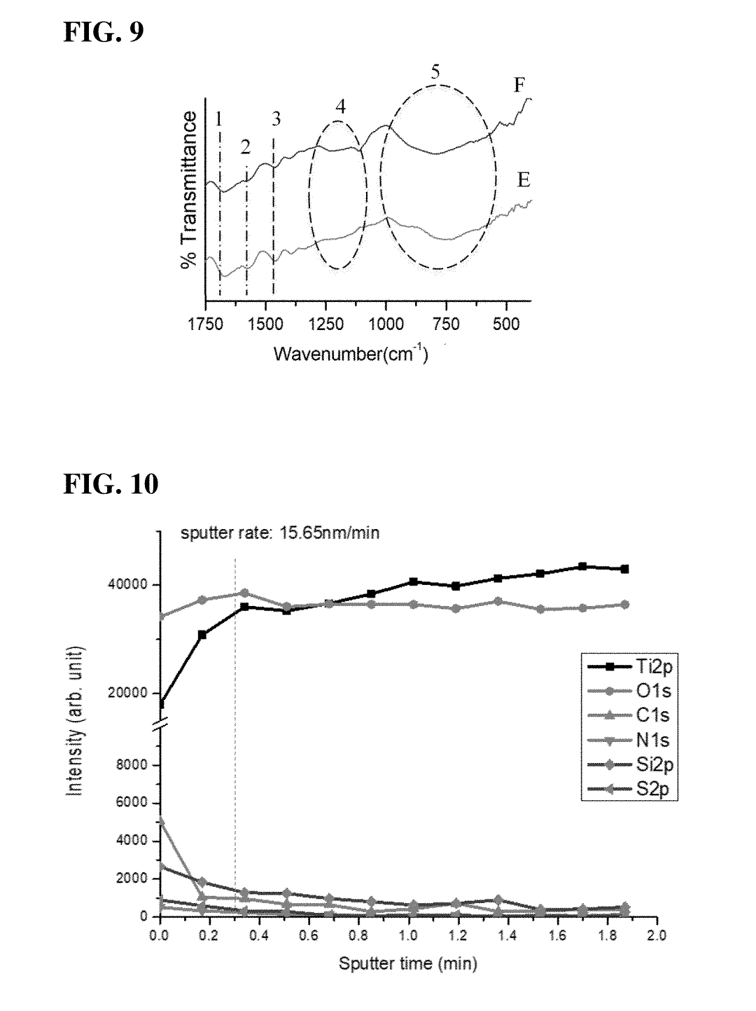

Then the modified substrates (samples E and F) produced in Examples 2 and 3, respectively, are analyzed using FTIR. FIG. 9 shows the peak 3 at 1,490 cm⁻¹ corresponding to the nitrate ions (NO₃⁻). After the samples were treated with N₂ and O₂ plasma, samples E and F show the distinct absorption peaks 1 and 2 at 1,680 and 1,565 cm⁻¹, attributed to the N—O bending vibration. The band 5 represents typical infrared absorption of Ti—O at 750 cm⁻¹ (broad, strong). The band 4 between 1,125 cm⁻¹ and 1,280 cm⁻¹ corresponds to the vibrations of —SO₃⁻ group. These are the results of the high content of negatively charged sulfur-containing and nitrogen-containing groups on the modified substrates.

According to the ESCA and FTIR spectra, the modified substrates produced from Examples 2 and 3 include sections having the structural formula (III) as the following:

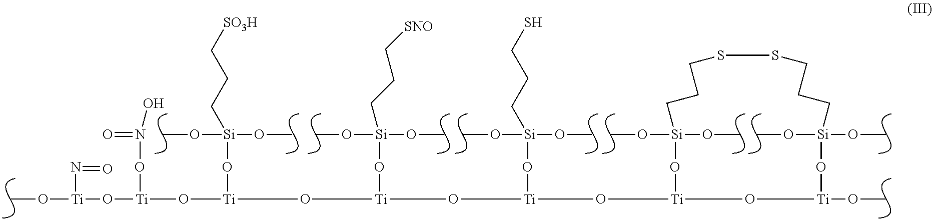

A common structural formula (II) of the present invention can be illustrated as the following:

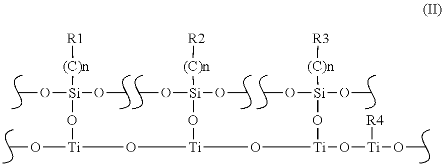

wherein

R1, R2 and R3 are each selected from the group consisting of —SO₃H, —SH and —SNO;

R4 is —NO or —NO₃H; and

(C)n is an alkyl chain comprising n carbon, n is an integer of 1-99.

Alternately, a more common structural formula (I) or (I′) can be illustrated as the following:

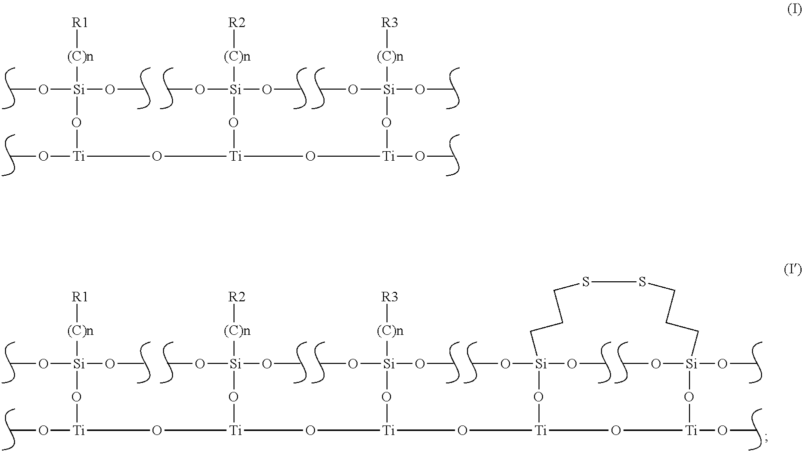

wherein

R1, R2 and R3 are each selected from the group consisting of —SO₃H, —SH and —SNO, and a disulfide bond (—S—S—) is further included in the structural formula (I′);

(C)n is an alkyl chain comprising n carbon, n is an integer of 1-99.

Alternately, a simpler structural formula (IV) can be illustrated as the following:

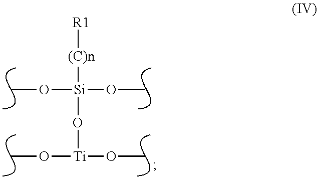

wherein

R1 is selected from the group consisting of —SO₃H, —SH and —SNO; and

(C)n is an alkyl chain, n is the carbon number in the range 1-99.

### Determining Thicknesses of the Modified Substrates

The modified substrates (samples E and F) produced in Examples 2 and 3 are analyzed using ESCA to analyze thicknesses thereof. FIG. 10 shows the depth profile of main atomic concentrations of the surface. The sputtering rate is 15.65 nm/min for 0.3 min so that the thickness is less than 5 nm, i.e. 4.695 nm.

Thickness of the modified substrate varies with the carbons number (n) of the alkyl chain. The carbon number can be in the range 1-100 and preferably 3-10, and therefore the thickness is in the range 0.1-100 nm and preferably 0.1-10 nm.

What is claimed is:

1. A surface modified structure of biomedical metallic substrate for improving hemocompatibility, having a section with a structural formula (I): 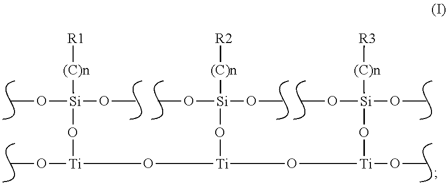
wherein
R1, R2 and R3 are each selected from the group consisting of —SO₃H, —SH and —SNO; and
(C)n is an alkyl chain comprising n carbon, n is an integer of 1-99.

2. The surface modified structure of claim 1, wherein R1 is —SO₃H.

3. The surface modified structure of claim 1, wherein n is an integer of 3-10.

4. The surface modified structure of claim 1, having a thickness of 0.1-100 nm.

5. The surface modified structure of claim 1, having a thickness of 0.1-10 nm.

6. The surface modified structure of claim 1, wherein the section further comprises a disulfide bond (—S—S—) and has a structural formula (I′): 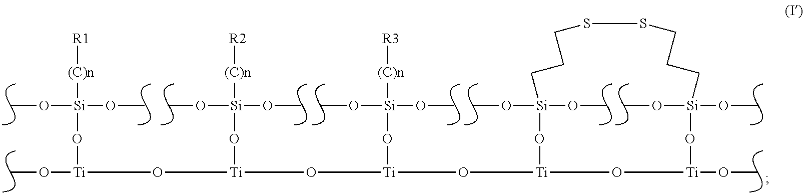
wherein
R1, R2, R3 and (C)n are defined as claim 1.

7. The surface modified structure of claim 1, wherein the section further comprises a functional group R4 and has a structural formula (II): 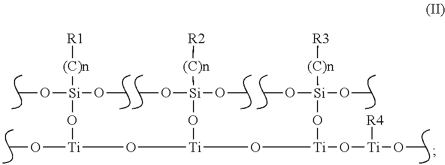
wherein
R1, R2, R3 and (C)n are defined as claim 1; and
R4 is —NO or —NO₃H.

8. The surface modified structure of claim 7, wherein R3 is —NO₃H.

9. A surface modified structure of biomedical metallic substrate for improving hemocompatibility of the biomedical metallic substrate, having a structural formula (III):

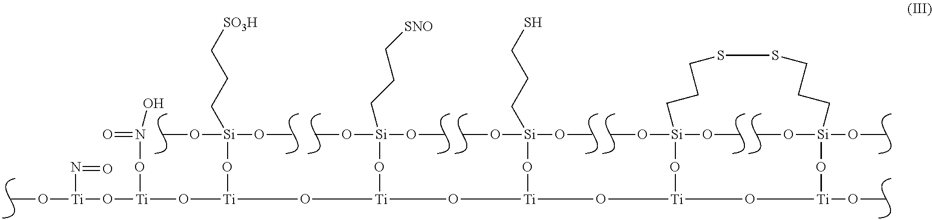

10. A surface modified structure of biomedical metallic substrate for improving hemocompatibility of the biomedical metallic substrate, having a structural formula (IV): 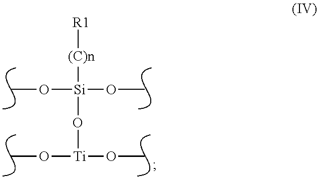
wherein
R1 is selected from the group consisting of —SO₃H, —SH and —SNO; and
(C)n is an alkyl chain, n is the carbon number in the range 1-99.
